(19) United States
(12) Patent Application Publication
(10) Pub. No.: US 2009/0069751 A1
(43) Pub. Date: Mar. 12, 2009

(54) Intravenous Delivery Systems

(76) Inventors: Brian E. Curtis, Chubbuck, ID (US); Zane L. Clement, Blackfoot, ID (US); Alan B. Curtis, Pocatello, ID (US)

Correspondence Address: FISH & RICHARDSON PC, P.O. BOX 1022, MINNEAPOLIS, MN, 55440-1022, US

(21) Appl. No.: 11/853,365

(22) Filed: Sep. 11, 2007

(51) Int. Cl. A61M 5/168; A61M 5/158

(52) U.S. Cl. 604/167.03; 604/164.01; 604/272

(57) ABSTRACT

An intravenous delivery system includes a device head and a needle carrier. The device head includes a body defining a first lumen extending from a first end to a second end and a second lumen in fluid communication with the first lumen and a port defined by the head body. A catheter disposed at the first end of the head body is in fluid communication with the first lumen. A self-sealing plug is disposed at the second end. The needle carrier is secured to the second end of the head body and includes a longitudinally extending housing and a hollow needle slidably disposed therein. A needle retractor coupled to the needle is operable to slide the needle longitudinally among deployed and retracted positions. The needle extends through the plug, first lumen, and catheter while in the deployed position, and is retracted into the needle carrier housing in the retracted position.

### TECHNICAL FIELD

This disclosure relates to intravenous delivery systems.

### BACKGROUND

Intravenous therapy or IV therapy is the delivery of liquid substances directly into a vein of a patient. The therapy can be intermittent or continuous. Continuous administration is generally called an intravenous drip. Compared with other routes of administration, intravenous therapy is generally the fastest way to deliver fluids and medications throughout the body of a patient.

A peripheral IV line includes a short catheter (a few centimeters long) inserted through the skin into a peripheral vein (e.g. any vein not in the chest or abdomen). Part of the catheter remains outside the skin with a hub that can be connected to a syringe or an intravenous infusion line, or capped with a bung between treatments.

### SUMMARY

In one aspect, an intravenous delivery system includes a device head and a needle carrier. The device head includes a head body having first and second ends. The head body defines a first lumen extending from the first end through the head body to the second end. The head body defines a second lumen in fluid communication with the first lumen and a port defined by the head body. A catheter disposed at the first end of the head body is in fluid communication with the first lumen. A self-sealing plug disposed at the second end of the head body inhibits fluid escapement from the first lumen at the second end of the head body. The needle carrier is secured to the second end of the head body of the device head. The needle carrier includes a longitudinally extending needle carrier housing and a needle disposed therein that defines a lumen extending therethrough. A needle retractor coupled to the needle is operable to slide the needle longitudinally among a deployed position and a retracted position. The needle pierces through the plug and extends through the first lumen and the catheter while in the deployed position. The needle is fully retracted into the needle carrier housing in the retracted position to allow decoupling of the needle carrier from the device head.

In another aspect, an intravenous delivery system head includes a head body having first and second ends. The head body defines a first lumen extending from the first end through the head body to the second end. The head body defines a second lumen in fluid communication with the first lumen and a port defined by the head body. A catheter disposed at the first end of the head body is in fluid communication with the first lumen. A self-sealing plug disposed at the second end of the head body inhibits fluid escapement from the first lumen at the second end of the head body. A valve is disposed in the second lumen of the head body and operable to restrict fluid flow though second lumen and out of the head body.

In yet another aspect, a needle carrier for an intravenous delivery system includes a longitudinally extending needle carrier housing, a needle defining a lumen extending therethrough slidably disposed in the needle carrier housing, and a needle retractor coupled to the needle. The needle retractor is operable to slide the needle longitudinally among a deployed position and a retracted position. The needle extends longitudinally out of the carrier housing in the deployed position and is fully retracted into the needle carrier housing in the retracted position.

In another aspect, a method of delivering intravenous therapy includes inserting a deployed needle of an intravenous delivery system into a patient. The intravenous delivery system includes a device head and a needle carrier. The device head includes a head body having first and second ends. The head body defines a first lumen extending from the first end through the head body to the second end. The head body defines a second lumen in fluid communication with the first lumen and a port defined by the head body. A catheter disposed at the first end of the head body is in fluid communication with the first lumen. A self-sealing plug disposed at the second end of the head body inhibits fluid escapement from the first lumen at the second end of the head body. The needle carrier is secured to the second end of the head body of the device head. The needle carrier includes a longitudinally extending needle carrier housing and a needle disposed therein that defines a lumen extending therethrough. A needle retractor coupled to the needle is operable to slide the needle longitudinally among a deployed position and a retracted position. The needle pierces through the plug and extends through the first lumen and the catheter while in the deployed position. The needle is fully retracted into the needle carrier housing in the retracted position to allow decoupling of the needle carrier from the device head. The method includes securing a fluid source to the port of the device head, such that the fluid source is in fluid communication with the second lumen, and actuating the needle retractor to move the needle to the retracted position, retracting the needle from the patient and the device head. With the needle retracted from the device head, fluid from the fluid source may flow through the second lumen into the first lumen and through the catheter into the patient.

In some implementations, the method includes decoupling the needle carrier from the device head. In some instances, the needle carrier is coupled to the device head via the needle inserted into and/or through the device head. In other instances, the needle carrier is coupled to the device head via a threaded coupling or another form of twist-on connection (e.g. a lure-lock). In some examples, after sliding the needle retractor to the retracted position, the needle retractor is locked in the retracted position to prevent redeployment of the needle to the deployed position.

In another aspect, a kit for intravenous therapy includes the combination of a device head and a needle carrier. The device head includes a head body having first and second ends. The head body defines a first lumen extending from the first end through the head body to the second end. The head body defines a second lumen in fluid communication with the first lumen and a port defined by the head body. A catheter disposed at the first end of the head body is in fluid communication with the first lumen. A self-sealing plug is disposed at the second end of the head body and inhibits fluid escapement from the first lumen at the second end of the head body. The needle carrier, for cooperative association with the second end of the head body of the device head, includes a longitudinally extending needle carrier housing and a needle defining a lumen extending therethrough. The needle is slidably disposed in the needle carrier housing. A needle retractor is coupled to the needle and is operable to slide the needle longitudinally among a deployed position and a retracted position. When the needle carrier is cooperatively associated with the second end of the head body of the device head, the needle pierces through the plug and extends through the first lumen and the catheter when in its deployed position. The needle is fully retracted into the needle carrier housing when in its retracted position to allow decoupling of the needle carrier from the device head.

Implementations of the disclosure may include one or more of the following features. In some implementations, the device head includes a valve disposed in the second lumen of the head body and operable to restrict fluid flow though second lumen and out of the head body. The valve includes a valve seat defined by the second lumen of the head body and a valve element disposed within the second lumen of the head body for movement between a first position in sealing engagement with the valve seat and a second position spaced from the valve seat for permitting fluid flow through the second lumen of the head body. The valve element defines a valve element operator. Contact with the valve element operator causes movement of the valve element from the first position to the second position, permitting fluid flow through the second lumen into the first lumen of the head body. In some instances, the valve element is urged toward sealing engagement with the valve seat by fluid pressure in the second lumen. In other instances, the valve element is urged toward sealing engagement with the valve seat by a biasing element (e.g. spring), or a combination of a biasing element and fluid pressure in the second lumen.

In some examples, the device head includes a catheter bushing disposed at the first end of the head body in the first lumen and sized to retain the catheter in fluid communication with the first lumen. The device head may include a fluid line connecter disposed at the port and in fluid communication with the second lumen. In some examples, the needle carrier includes a plug vent in fluid communication with the needle and operable to purge gases from the needle.

The needle retractor may be lockable in the retracted position to prevent redeployment of the needle to the deployed position. The needle carrier defines a locking feature that receives and locks the needle retractor in the retracted position. In some instances, the needle retractor includes a graspable retractor body slidably disposed on the needle carrier housing for sliding the needle among the deployed position and the retracted position. In other instances, the needle retractor includes a needle retainer temporarily holding the needle in the deployed position and a retractor spring disposed in the needle carrier housing and biasing the needle toward the retracted position. The spring moves the needle to the retracted position when released from the deployed position.

The details of one or more implementations of the disclosure are set fourth in the accompanying drawings and the description below. Other features, objects, and advantages will be apparent from the description and drawings, and from the claims.

### DESCRIPTION OF DRAWINGS

FIG. 1 is a perspective view of an intravenous delivery system.

FIG. 2 is a perspective view of a device head of an intravenous delivery system.

FIG. 3 is a section view of the device head of FIG. 2.

FIG. 4 is a perspective view of a needle carrier of an intravenous delivery system.

FIG. 5 is a side view of a needle carrier of an intravenous delivery system.

FIG. 6 is a section view of the needle carrier of FIG. 5.

FIG. 7 is a side perspective view of a plug vent of a needle carrier of an intravenous delivery system.

FIG. 8 is a side perspective view of a needle of a needle carrier of an intravenous delivery system.

FIG. 9 is a top perspective view of an intravenous delivery system.

FIG. 10 is a side perspective view of an intravenous delivery system.

FIG. 11 is an exploded view of an intravenous delivery system.

FIG. 12 is a section view of an intravenous delivery system.

Like reference symbols in the various drawings indicate like elements.

### DETAILED DESCRIPTION

Paramedics, nurses, and other medical personnel administer intravenous therapy to patients on a routine basis. Current intravenous delivery systems employ the use of a syringe pierced though a hub joined to a catheter to insert the catheter into a vein of a patient. After placement of the catheter into the vein, the syringe is removed from the catheter and hub for subsequent delivery of a fluid through the catheter. Current intravenous delivery systems allow blood to spill out of the hub after removal of the syringe, thereby creating a potential hazard for blood-born pathogens. The removed syringe must also be safety disposed. Accidental injury from the syringe, either during use or from a discarded one, poses significant health threats (e.g. the spread of HIV, hepatitis, etc.). The disclosed intravenous delivery system advantageously provides a way of safely administering intravenous therapy without significant exposure to blood or used needles.

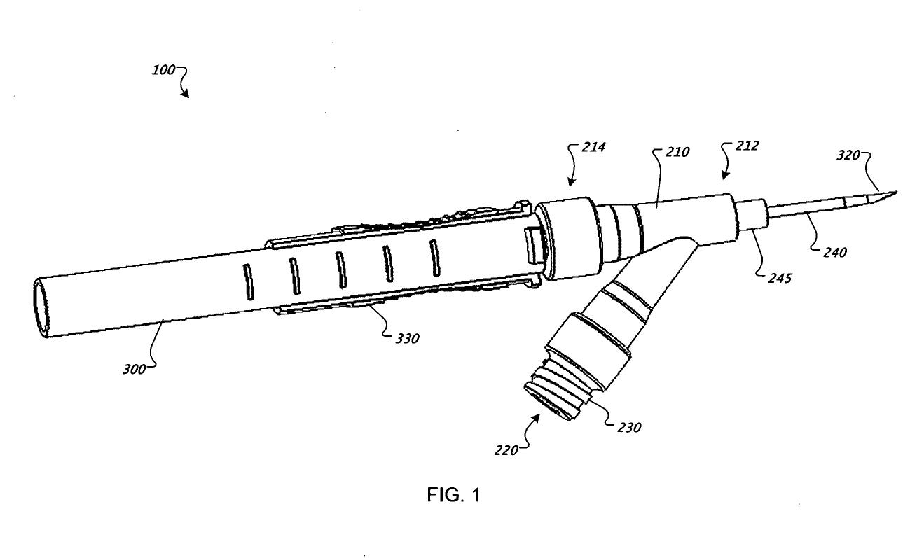

Referring to FIGS. 1-3, an intravenous delivery system 100 includes a device head 200 and a needle carrier 300. The device head 200 includes a head body 210 having first and second ends 212 and 214, respectively. The head body 210 defines a first lumen 215 extending from the first end 212 through the head body 210 to the second end 214. The head body 210 defines a second lumen 217 in fluid communication with the first lumen 215 and a port 220 defined by the head body 210. The first lumen 215 is substantially straight, and the second lumen 217 branches off the first lumen 215 and may be non-linear. In some implementations, the head body 210 is an integrally molded piece. In other implementations, the head body 210 includes a rigid or flexible branch tube 225 disposed on the head body 210 and defining the second lumen 217 in fluid communication with the first lumen 215 and the port 220. The branch tube 225 may be about 10 cm long and disposed on the head body 210, forming a “Y” configuration. A fluid line connecter 230 is disposed at the port 220 and in fluid communication with the second lumen 217. The fluid line connecter 230 is secured to or defined by the head body 210 or branch tube 225 at the port 220 and provides a connection interface for the device head 200. A valve 260 is disposed at the port 220 or in the second lumen 217 and is configured to prevent fluid flow out of the head body 210 through the port 220, while allowing fluid flow into the head body 210 through the second lumen 217. For example, the valve 260 prevents blood flow out of the device head 200, while permitting the delivery of fluids through the device head 200 into a patient. In some implementations, the valve 260 includes a valve seat 262 defined by the second lumen 217 near the port 220. The valve seat 262 may be a lip defined by the second lumen 217 at the port 220 or a side wall portion of the second lumen 217. A valve element 264 is disposed within the second lumen 217 for movement among a first position in sealing engagement with the valve seat 262 and a second position spaced from the valve seat 262, permitting fluid flow through the second lumen 217 and into the first lumen 215 (for delivery to a patient). A valve element operator 266 (in one example, a portion of the valve element 264) is operable for actuating engagement by a mating connector of the fluid line connector 230. The valve element 320 may be spherical, elliptical, cylindrical, cubical, pyramidal, or any other suitable shape. The valve element operator 266 may be a portion of the valve element 264 protruding outward toward the port 220, extending through the second lumen 217 past the valve seat 262.

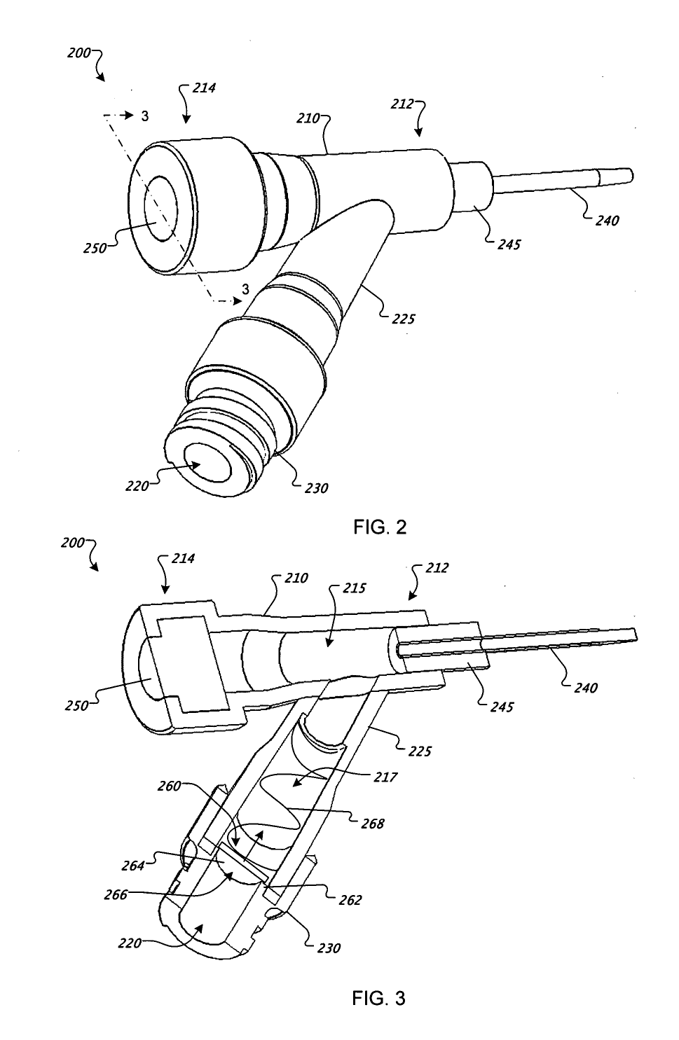

The valve element 264 may be urge toward its first position in sealing engagement with the valve seat by fluid pressure in the head body 210. Alternatively, in the examples of FIGS. 3 and 12, the valve 260 includes a spring 268 (which may extend integrally from the valve element 264) biasing the valve element 264 into sealing engagement with the valve seat 262, causing the valve 260 to remain closed while not actuated. A combination of fluid pressure and biasing element may also be employed.

A catheter 240 is disposed at the first end 212 of the head body 210 in fluid communication with the first lumen 215. In some implementations, the device head 200 includes a catheter bushing 245 disposed at the first end 212 of the head body 210 in the first lumen 215 and sized to retain the catheter 240 in fluid communication with the first lumen 215. The catheter bushing 245 is sized according to the catheter 240 used and provides an interface between the head body 210 and the catheter 240. A self-sealing plug 250 is disposed at the second end 214 of the head body 210. The self-sealing plug 250 inhibits fluid from escaping from the first lumen 215 at the second end 214 of the head body 210. The self-sealing plug 250 (e.g. a surgical rubber) is configured to be pierced by needles (e.g. syringes) and then self-seal any holes created by a subsequently removed needle.

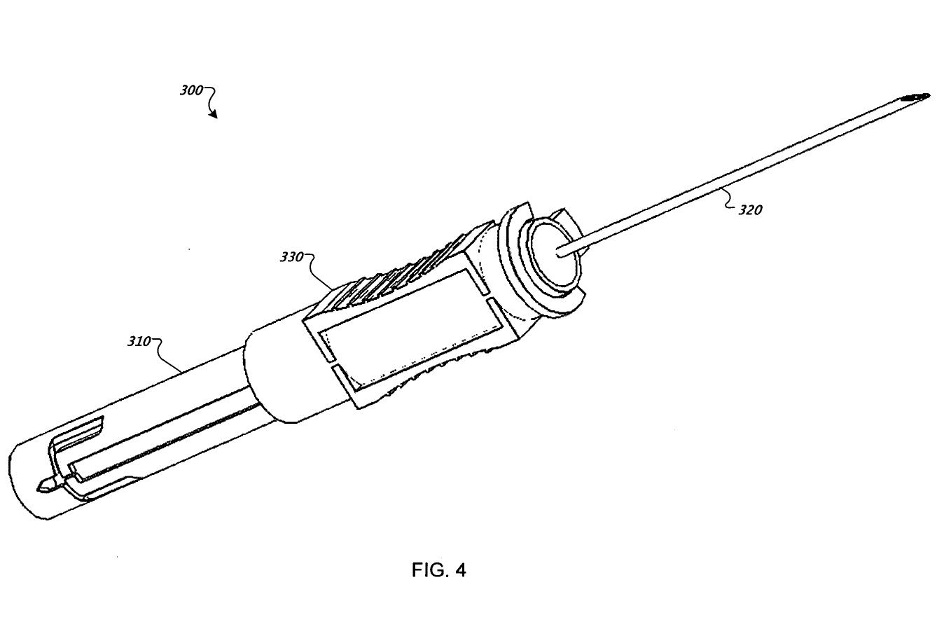

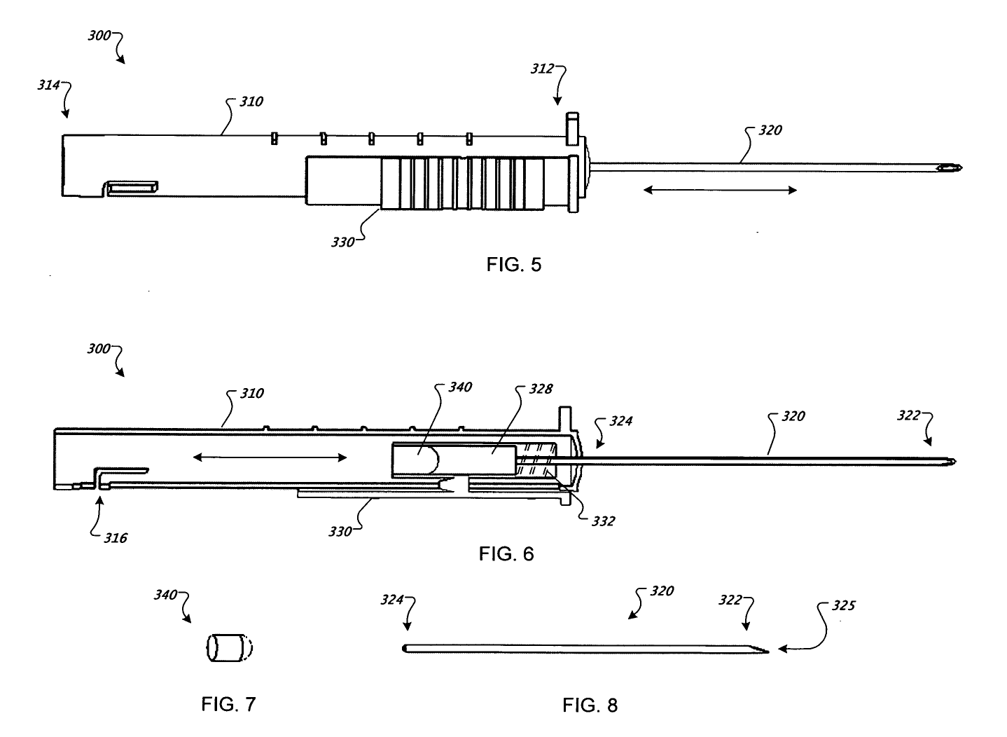

Referring to FIGS. 4-8, the needle carrier 300 includes a longitudinally extending needle carrier housing 310 having first and second ends 312 and 314, respectively. A needle 320 having first and second ends 322 and 324, respectively, and defining a lumen 325 extending therethrough is slidably disposed in the needle carrier housing 310. A needle retractor 330 coupled to the needle 320 is operable to slide the needle 320 longitudinally among a deployed position and a retracted position. The needle retractor may be a graspable body slidably disposed on the needle carrier housing 310 for sliding the needle 330 among the deployed position and the retracted position. The needle 320 is shown in the deployed position in the examples illustrated in FIGS. 4-6. In the retracted position, the needle 320 is fully retracted into the carrier housing 310 to protect a user from the ends 322, 324 of the needle 320. Retracting the needle 320 to the retracted position inside the needle carrier housing 310, especially after use, protects users from accidental injury by the needle 320. The needle carrier 300 may include a plug vent 340 in fluid communication with the second end 324 of the needle 320 and operable to purge gases from the needle 320. For example, the plug vent 340 allows air in the needle 320 to be purged as blood from a patient stuck by the needle 320 enters the needle 320. The plug vent 340 prevents the blood from escaping the needle 320. In some examples, a reservoir 328 in fluid communication with the second end 324 of the needle 320 houses the plug vent 340. The reservoir 328 receives fluid and gases passing through the needle 320. The plug vent 340 allows the gases to be purged from the reservoir 328.

In some implementations, the needle retractor 330 is lockable in the retracted position to prevent redeployment of the needle 320 to the deployed position. The needle carrier housing 310 may define a locking feature 316 that receives and locks the needle retractor 330 in the retracted position. The locking feature 316 may be a compliant or deformable lip. As the needle retractor 330 slides over the feature 316, the feature 316 temporarily deforms and then resiliently returns to be received by a corresponding mating feature or recess defined by the needle retractor 330. The received feature 316 prevents subsequent movement of the needle retractor 330 and thereby prevents redeployment of the needle 320 to the deployed position.

In some implementations, the needle retractor 330 acts as a needle retainer temporarily holding the needle 320 in the deployed position. A retractor spring 332 disposed in the needle carrier housing 310 biases the needle 320 toward the retracted position. The spring 332 moves or urges the needle 320 to the retracted position when a user releases the needle 320 from the needle retainer 330 and the deployed position.

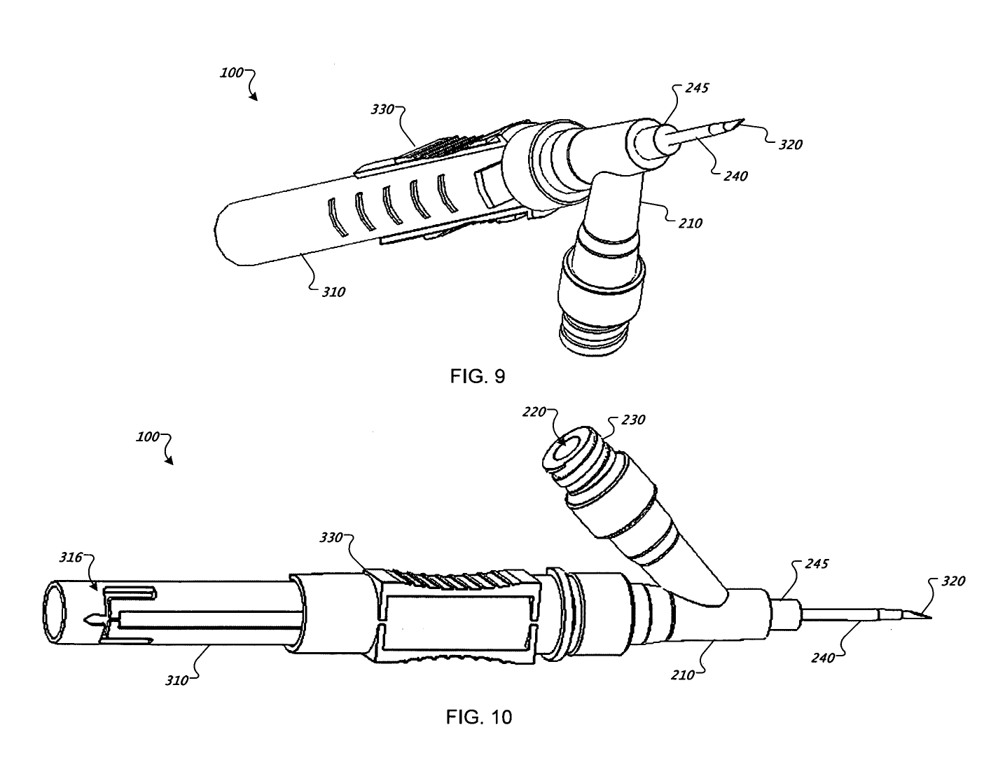

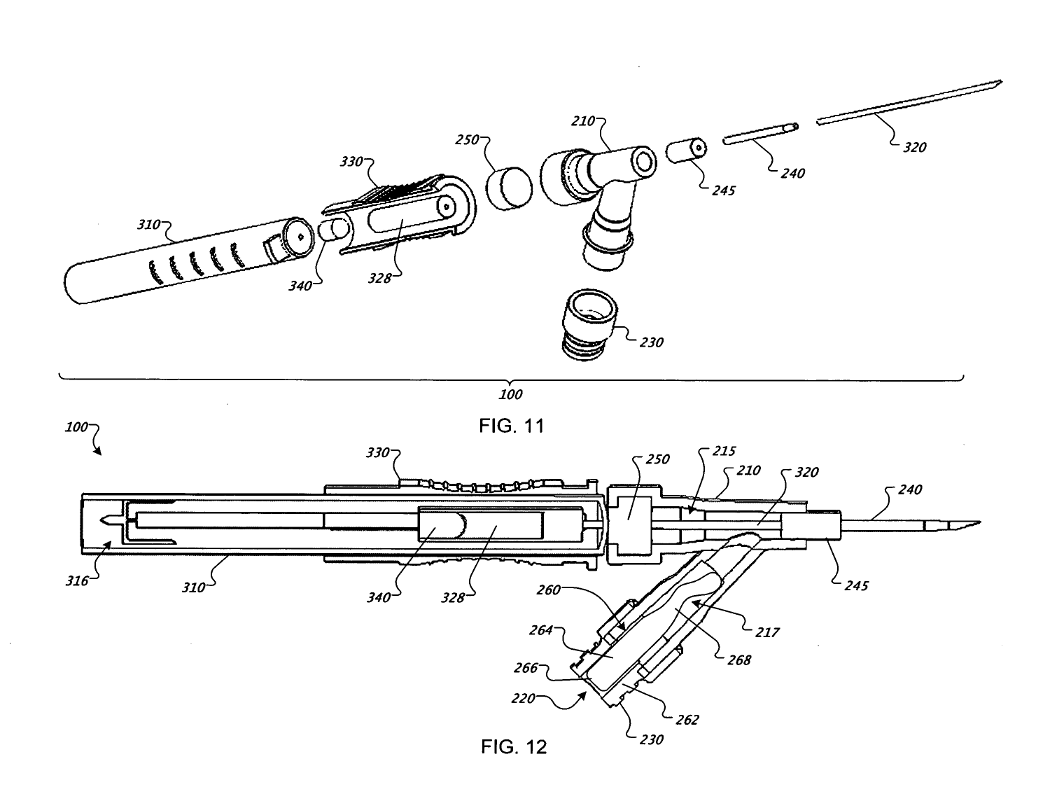

In the examples illustrated in FIGS. 9-12, the intravenous delivery system 100 includes the needle carrier 300 with the needle 320 in the deployed position piercing through the plug 250 of the device head 200 and extending through the first lumen 215 of the head body 210 as well as the catheter 240, ready for application to a patient. A method of delivering intravenous therapy includes inserting the deployed needle 320 of the intravenous delivery system 100 into a patient, securing a fluid source (e.g. intravenous fluid bag) to the port 220 of the device head 200, such that the fluid source is in fluid communication with the second lumen 217 of the head body 210, and actuating the needle retractor 330 to move the needle 320 to the retracted position, thereby retracting the needle from the patient and the device head 200. Retracting the needle 320 fully into the needle carrier housing 310 in the retracted position decouples the needle carrier 300 from the device head 200. In some examples, the needle carrier 300 is coupled to the second end 214 of the device head 200 (e.g. via a threaded interface) and must be decoupled from the device head 200 after the needle 320 is moved to the retracted position. The fluid source may be connected to the port 220 via a fluid line connector 230 secured to the head body 210 and in fluid communication with the second lumen 217.

The intravenous delivery system 100 may be provided as a kit including the device head 200 and the needle carrier 300 together or packaged separately. When provided as a kit, a user actuates the needle 320 of the needle carrier 300 to the deployed position (if not provided in the deployed position) and inserts the needle 320 into the second end 214 of the device head 200 by piecing the plug 250. The needle 320 is pushed though the first lumen 215 of the head body 210 and the catheter 240, exposing the first end 322 (e.g. tip) of the needle 320 past the catheter 240. The intravenous delivery system 100 with the deployed needle 320 passing through the device head 200 is ready for administration to a patient. Administration of the intravenous delivery system 100 includes insertion of the deployed needle 320 into the vein of a patient to thereby set the catheter 240 in the vein. After setting the catheter 240 in the vein, the needle 320 is retracted into the needle carrier 300 via the needle retractor 330. Upon retracting the needle 320, the needle carrier 300 is disjoined or disconnected from the device head 200 and discarded. The device head 200 provides two sites for delivering intravenous therapy to the patient. An intravenous delivery line may be attached to the port 220, in fluid communication with the second lumen 217, and a syringe may be pierced through the plug 250 to inject fluid into the first lumen 215. When the intravenous therapy is complete, the catheter 240 is removed from the patient's vein and the device head 200 is safely discarded.

A number of implementations have been described. Nevertheless, it will be understood that various modifications may be made without departing from the spirit and scope of the disclosure. Accordingly, other implementations are within the scope of the following claims.

What is claimed is:

1. An intravenous delivery system comprising:
a device head comprising:
a head body having first and second ends, the head body defining a first lumen extending from the first end through the head body to the second end, the head body defining a second lumen in fluid communication with the first lumen and a port defined by the head body;
a catheter disposed at the first end of the head body in fluid communication with the first lumen; and
a self-sealing plug disposed at the second end of the head body and inhibiting fluid escapement from the first lumen at the second end of the head body; and
a needle carrier secured to the second end of the head body of the device head, the needle carrier comprising:
a longitudinally extending needle carrier housing;
a needle defining a lumen extending therethrough, the needle slidably disposed in the needle carrier housing; and
a needle retractor coupled to the needle, the needle retractor operable to slide the needle longitudinally among a deployed position and a retracted position;
wherein the needle pierces through the plug and extends through the first lumen and the catheter while in the deployed position, and wherein the needle is fully retracted into the needle carrier housing in the retracted position to allow decoupling of the needle carrier from the device head.

2. The intravenous system of claim 1, wherein the device head further comprises a valve disposed in the second lumen of the head body and operable to restrict fluid flow though second lumen and out of the head body.

3. The intravenous system of claim 2, wherein the valve comprises:
a valve seat defined by the second lumen of the head body; and
a valve element disposed within the second lumen of the head body for movement between a first position in sealing engagement with the valve seat and a second position spaced from the valve seat for permitting fluid flow through the second lumen of the head body, the valve element defining a valve element operator;
wherein contact with the valve element operator causes movement of the valve element from the first position to the second position, permitting fluid flow through the second lumen into the first lumen of the head body.

4. The intravenous system of claim 3, wherein the valve element is urged toward sealing engagement with the valve seat by a biasing element.

5. The intravenous system of claim 1, wherein the needle carrier further comprises a plug vent in fluid communication with the needle and operable to purge gases from the needle.

6. The intravenous system of claim 1, wherein the needle retractor is lockable in the retracted position to prevent redeployment of the needle to the deployed position.

7. The intravenous system of claim 6, wherein the needle carrier defines a locking feature that receives and locks the needle retractor in the retracted position.

8. The intravenous system of claim 1, wherein the device head further comprises a fluid line connecter disposed at the port and in fluid communication with the second lumen, the fluid line connecter configured to receive a corresponding mating connector of a fluid delivery line.

9. An intravenous delivery system head comprising:
a head body having first and second ends, the head body defining a first lumen extending from the first end through the head body to the second end, the head body defining a second lumen in fluid communication with the first lumen and a port defined by the head body;
a catheter disposed at the first end of the head body in fluid communication with the first lumen;
a self-sealing plug disposed at the second end of the head body and inhibiting fluid escapement from the first lumen at the second end of the head body; and
a valve disposed in the second lumen of the head body and operable to restrict fluid flow though second lumen and out of the head body.

10. The intravenous delivery system head of claim 9, wherein the valve comprises:
a valve seat defined by the second lumen of the device head; and
a valve element disposed within the second lumen of the head body for movement between a first position in sealing engagement with the valve seat and a second position spaced from the valve seat for permitting fluid flow through the second lumen of the head body, the valve element defining a valve element operator;
wherein contact with the valve element operator causes movement of the valve element from the first position to the second position, permitting fluid flow through the second lumen into the first lumen of the head body.

11. The intravenous system of claim 10, wherein the valve element is urged toward sealing engagement with the valve seat by a biasing element.

12. A needle carrier for an intravenous delivery system, the needle carrier comprising:
a longitudinally extending needle carrier housing;
a needle defining a lumen extending therethrough, the needle slidably disposed in the needle carrier housing;
a plug vent in fluid communication with the needle and operable to purge gases from the needle; and
a needle retractor coupled to the needle, the needle retractor operable to slide the needle longitudinally among a deployed position and a retracted position;
wherein the needle extends longitudinally out of the carrier housing in the deployed position and is fully retracted into the needle carrier housing in the retracted position;
wherein the needle retractor is lockable in the retracted position to prevent redeployment of the needle to the deployed position.

13. The needle carrier of claim 12, wherein the needle carrier defines a locking feature that receives and locks the needle retractor in the retracted position.

14. A method of delivering intravenous therapy, the method comprising:
inserting a deployed needle of an intravenous delivery system into a patient, the intravenous system comprising:
a device head comprising:
a head body having first and second ends, the head body defining a first lumen extending from the first end through the head body to the second end, the head body defining a second lumen in fluid communication with the first lumen and a port defined by the head body;
a catheter disposed at the first end of the head body in fluid communication with the first lumen; and
a self-sealing plug disposed at the second end of the head body and inhibiting fluid escapement from the first lumen at the second end of the head body; and
a needle carrier secured to the second end of the head body of the device head, the needle carrier comprising:
a longitudinally extending needle carrier housing;
the needle defining a lumen extending therethrough, the needle slidably disposed in the needle carrier housing; and
a needle retractor coupled to the needle, the needle retractor operable to slide the needle longitudinally among a deployed position and a retracted position;
wherein the needle pierces through the plug and extends through the first lumen and the catheter while in the deployed position, and wherein the needle is fully retracted into the needle carrier housing in the retracted position to allow decoupling of the needle carrier from the device head;
securing a fluid source to the port of the device head, the fluid source being in fluid communication with the second lumen; and
actuating the needle retractor to move the needle to the retracted position, retracting the needle from the patient and the device head.

15. The method of claim 14, wherein the device head further comprises a valve disposed in the second lumen of the head body and operable to restrict fluid flow though second lumen and out of the head body.

16. The intravenous system of claim 15, wherein the valve comprises:
a valve seat defined by the second lumen of the head body; and
a valve element disposed within the second lumen of the head body for movement between a first position in sealing engagement with the valve seat and a second position spaced from the valve seat for permitting fluid flow through the second lumen of the head body, the valve element defining a valve element operator;
wherein contact with the valve element operator causes movement of the valve element from the first position to the second position, permitting fluid flow through the second lumen into the first lumen of the head body.

17. The method of claim 16, wherein the valve element is urged toward sealing engagement with the valve seat by a biasing element.

18. The method of claim 14, wherein the needle carrier further comprises a plug vent in fluid communication with the needle and operable to purge gases from the needle.

19. The method of claim 14, wherein after sliding the needle retractor to the retracted position, the needle retractor is locked in the retracted position to prevent redeployment of the needle to the deployed position.

20. The method of claim 14, wherein the device head further comprises a fluid line connecter disposed at the port and in fluid communication with the second lumen, the fluid line connecter configured to receive a corresponding mating connector of a fluid delivery line.

21. A kit for intravenous therapy, the kit comprising the combination of:
a device head comprising:
a head body having first and second ends, the head body defining a first lumen extending from the first end through the head body to the second end, the head body defining a second lumen in fluid communication with the first lumen and a port defined by the head body;
a catheter disposed at the first end of the head body in fluid communication with the first lumen; and
a self-sealing plug disposed at the second end of the head body and inhibiting fluid escapement from the first lumen at the second end of the head body; and
a needle carrier for cooperative association with the second end of the head body of the device head, the needle carrier comprising:
a longitudinally extending needle carrier housing;
a needle defining a lumen extending therethrough, the needle slidably disposed in the needle carrier housing; and
a needle retractor coupled to the needle, the needle retractor operable to slide the needle longitudinally among a deployed position and a retracted position;
wherein when cooperatively associated with the second end of the head body of the device head, the needle pierces through the plug and extends through the first lumen and the catheter when in its deployed position, and wherein the needle is fully retracted into the needle carrier housing when in its retracted position to allow decoupling of the needle carrier from the device head.

22. The kit for intravenous therapy of claim 21, wherein the device head further comprises a valve disposed in the second lumen of the head body and operable to restrict fluid flow though second lumen and out of the head body.

23. The kit for intravenous therapy of claim 22, wherein the valve comprises:
a valve seat defined by the second lumen of the head body; and
a valve element disposed within the second lumen of the head body for movement between a first position in sealing engagement with the valve seat and a second position spaced from the valve seat for permitting fluid flow through the second lumen of the head body, the valve element defining a valve element operator;
wherein contact with the valve element operator causes movement of the valve element from the first position to the second position, permitting fluid flow through the second lumen into the first lumen of the head body.

24. The kit for intravenous therapy of claim 23, wherein the valve element is urged toward sealing engagement with the valve seat by a biasing element.

25. The kit for intravenous therapy of claim 21, wherein the device head further comprises a fluid line connecter disposed at the port and in fluid communication with the second lumen, the fluid line connecter configured to receive a corresponding mating connector of a fluid delivery line.
